(19) United States
(12) Patent Application Publication
(10) Pub. No.: US 2022/0177853 A1
(43) Pub. Date: Jun. 9, 2022

(54) METHODS FOR CONTROLLED INDUCTION OF BIOENGINEERED NEUROEPITHELIAL TISSUES AND 3-D NEUROEPITHELIAL TUBES

(71) Applicant: Wisconsin Alumni Research Foundation, Madison, WI (US)

(72) Inventors: Randolph Scott Ashton, Madison, WI (US); Gavin Thomas Knight, Madison, WI (US); Benjamin John Knudsen, Menomonee Falls, WI (US); Nisha Ramdas Iyer, Madison, WI (US); Carlos Marti-Figueroa, Madison, WI (US)

(21) Appl. No.: 17/545,416

(22) Filed: Dec. 8, 2021

Related U.S. Application Data
(60) Provisional application No. 63/122,918, filed on Dec. 8, 2020.
(60) Provisional application No. 63/127,974, filed on Dec. 18, 2020.

(51) Int. Cl. C12N 5/071; A61L 27/36; C12N 5/079

(52) U.S. Cl. CPC C12N 5/0697; A61L 27/3687; C12N 5/0618; A61L 27/3675; A61L 27/3604; C12N 2501/41; C12N 2506/02; C12N 2533/30; C12N 2531/00; C12N 2535/10; C12N 2501/999

(57) ABSTRACT

Described herein are methods, compositions, and kits for directed differentiation of human pluripotent stem cells, neuromesodermal progenitors, and neural stem cells into bioengineered elliptical neuroepithelial tissues and bioengineered neuroepithelial tubes that contain a single rosette of polarized neuroepithelial cells and have microscale cellular organization similar to that of an in vivo developing human neural tube.

### STATEMENT REGARDING FEDERALLY SPONSORED RESEARCH OR DEVELOPMENT

This invention was made with government support under RD-83573701-0 awarded by the Environmental Protection Agency and under NS082618 awarded by the National Institutes of Health and under 1651645 awarded by the National Science Foundation. The government has certain rights in the invention.

### BACKGROUND

Human pluripotent stem cells (hPSCs) provide an unlimited starting material for biomanufacturing tissue/organ transplants and engineering a wide variety of tissue, organ, and embryo models in vitro to investigate human development, physiology, and disease. Much progress has been made using suspension embryoid body culture approaches to generate in vitro models of brain, kidney, liver, prostate, and even gastrulating embryo-like tissues; however, their structural and compositional variability limits the use of organoids as a scalable in vitro screening platform or for tissue/organ transplant biomanufacturing.

The brain and spinal cord develop from the neural tube, a hollow tube of polarized neuroepithelial cells (also termed neural stem cells). In vitro, 2- and 3-D cultures of differentiating neuroepithelial cells can initiate organoid formation through spontaneous polarization of neural rosettes, which function like slices of the neural tube. Neural stem cell differentiation protocols have been previously disclosed as well as a micropatterned in vitro culture protocol for controlled formation of singular neural rosette structures. Such neural rosettes model a transverse slice of the developing neural tube, but their small size limits their use as a substrate for further bioengineering; for example, the diameter is too small to generate a significant microfluidic gradient of cell culture factors. Thus, there is an ongoing need for improved methods and compositions for controlled induction of formation of larger rosette tissues that are more biomimetic of neural tube morphology and that facilitate further morphogenetic bioengineering using microfluidic platforms.

### SUMMARY

Described herein are methods, compositions, and kits for making larger bioengineered neuroepithelial tissues and neuroepithelial tubes that are more biomimetic of neural tube morphology and facilitate further morphogenetic bioengineering using microfluidic platforms.

In a first aspect, provided herein are in vitro methods for efficiently, reproducibly, and robustly producing bioengineered neuroepithelial tissues having a singular neuroepithelial rosette core, where the tissue has an elliptical morphology (e.g., major axis up to >1 mm and minor axis about 250-300 μm) that can be cultured further to form an 3D tubular structure having characteristics of a nascent neural tube, from biomimetic elliptical neuroepithelial tissues.

In certain embodiments, provided herein are methods for producing biomimetic elliptical neuroepithelial tissue having a singular rosette structure in vitro that comprises the following steps: (a) seeding human pluripotent stem cells (hPSCs) in the presence of a Rho kinase inhibitor onto a micropatterned substrate that induces biomimetic neural morphogenesis of cells cultured thereon, wherein the micropatterned substrate comprises at least two cell-adhesive, circular regions connected by a cell-adhesive bridge; (b) culturing the seeded micropatterned substrate for a first culture period of about one to two days in the presence of a pluripotency maintenance base medium to obtain a first cell population, wherein the pluripotency maintenance base medium comprises a Rho kinase inhibitor; (c) culturing the micropatterned substrate thereof for a second period of about 2 to about 6 days under adherent conditions in a neural differentiation base medium, whereby a biomimetic elliptical neuroepithelial tissue comprising polarized neuroepithelial cells and having microscale cellular organization similar to that of a transverse section of an in vivo developing human neural tube is obtained; and (d) overlaying the adherent micropatterned culture with an hydrogel from about day 5 to about day 7 of the second culture period to release the elliptical neuroepithelial tissue from the substrate allowing it to become a 3D neuroepithelial tissue while maintaining a singularly polarized annular structure, akin to the in vivo developing human neural tube.

In certain embodiments, the above methods further comprise overlaying the bioengineered neuroepithelial tissue obtained therein with a hydrogel layer, culturing the bioengineered neuroepithelial tissue for about 24 hours, whereby the tissue morphs into a bioengineered neuroepithelial tube and becomes encased in the hydrogel layer, and removing the hydrogel comprising the encased neuroepithelial tube from the micropatterned substrate, to obtain a bioengineered neuroepithelial tube.

In another aspect, therefore, this disclosure provides is a microfluidics device comprising a micropatterned substrate as described herein for obtaining an in vitro bioengineered neuroepithelial tissue. As used herein, the term “morphogen” refers to a molecule or mixture of molecules that induces differentiation and/or proliferation of a cell such as a neural progenitor. In one embodiment, the morphogen provides spatial information via a concentration gradient that can affect patterning of a differentiating neural tissue. In some embodiments, a morphogen is a diffusible protein, cytokine, or growth factor.

In another aspect, provided herein is a kit comprising one or more components useful for obtaining an in vitro biomimetic neuroepithelial tissue or a bioengineered neuroepithelial tube. Components of the kit can include one or more micropatterned substrates as described herein. The kit can also contain a chemically defined culture medium and one or more medium additives (e.g., growth factors, small molecule compounds, culture medium supplements). The kit can further contain progenitor cells useful for the seeding of the micropatterned substrate and instructions for cell seeding and culture. In some embodiments, it will be advantageous for a kit to comprise one or more micropatterned substrates as described herein and one or more agents (e.g., growth factors, small molecule agents, agents to create a morphogen gradient) useful to further differentiate the neuroepithelial tissue to contain various types of neurons. In such embodiments, the kit can also comprise a microfluidics device or components thereof, for example, to deliver patterning agents to the bioengineered neuroepithelial tissue in a pre-determined manner. In some embodiments, the kit further comprises a hydrogel suitable for use as an overlay on the biomimetic elliptical neuroepithelial tissue to obtain an encased bioengineered neuroepithelial tube according to methods of this disclosure.

In another aspect, provided herein is engineered in vitro bioengineered neuroepithelial tissue obtained by one of the methods disclosed herein, comprising a single rosette of polarized neuroepithelial cells and having microscale cellular organization similar to that of a transverse section of an in vivo developing human neural tube.

In another aspect, provided herein is a composition comprising one or more micropatterned substrates that is capable of biomimetic neural morphogenesis of cells cultured thereon, wherein the micropatterned substrate comprises at least two circular, cell-adhesive microscale regions and connected by a cell-adhesive bridge.

In a first particular aspect, the present disclosure provides a method of producing a biomimetic elliptical neuroepithelial tissue having a singular rosette structure in vitro. The method includes: (a) seeding human pluripotent stem cells (hPSCs) in the presence of a Rho kinase inhibitor onto a micropatterned substrate that is capable of biomimetic neural morphogenesis of cells cultured thereon, wherein the micropatterned substrate comprises at least two circular bounded regions connected by a cell-adhesive bridge; (b) culturing the seeded cells of step (a) on the micropatterned substrate for a first culture period of about one to two days in the presence of a pluripotency maintenance medium to obtain a first cell aggregate, wherein the pluripotency maintenance medium comprises a Rho kinase inhibitor; and (c) culturing the cells obtained in step (b) for a second culture period of about 3 to about 6 days under adherent culture conditions in a neural differentiation base medium, whereby a biomimetic elliptical neuroepithelial tissue having a singular rosette structure is obtained. The biomimetic elliptical neuroepithelial tissue comprises polarized neuroepithelial cells and has a microscale cellular organization similar to that of a transverse section of an in vivo developing human neural tube.

In one embodiment of the first particular aspect, each of the at least two circular bounded regions has a diameter of about 100 μm to about 300 μm. In one embodiment of the first particular aspect, the cell-adhesive bridge has a length of about 25 μm to about 125 μm, and has a width of about 10 μm to about 50 μm. In one embodiment of the first particular aspect, the hPSCs are seeded onto the micropatterned substrate at a density of between about 75×10³ cells/cm² and about 2.5×10⁵ cells/cm². In one embodiment of the first particular aspect, the pluripotency maintenance medium is a chemically defined medium comprising DMEM/F-12, ascorbic acid, sodium bicarbonate, selenium, insulin, transferrin, FGF2, and TGFβ1. In one embodiment of the first particular aspect, the pluripotency maintenance medium is E8 medium.

In one embodiment of the first particular aspect, the neural differentiation base medium is a chemically defined medium comprising DMEM/F-12, ascorbic acid, sodium bicarbonate, selenium, insulin, and transferrin. In one embodiment of the first particular aspect, the neural differentiation base medium is E6 medium. In one embodiment of the first particular aspect, the neural differentiation base medium further comprises one or more of an FGF with or without an activator of β-catenin pathway signaling.

In one embodiment of the first particular aspect, the FGF is FGF2, FGF8a, FGF8b, FGF8f, FGF17, or FGF18. In one embodiment of the first particular aspect, the activator of β-catenin pathway signaling is a GSK3 kinase inhibitor. In one embodiment of the first particular aspect, the GSK3 kinase inhibitor is CHIR99021.

In one embodiment of the first particular aspect, the method further comprises transiently exposing cells on the micropatterned substrate to an activator of Wnt/β-catenin signaling about 24-72 hours after plating onto the micropatterned substrate.

In one embodiment of the first particular aspect, the method further comprises about 24-72 hours after seeding cells onto the micropatterned substrate, exposing the seeded cells to RA and Sonic Hedgehog (SHH) or a SHH signaling agonist for about 1 to about 5 days, whereby the rosette structure comprises Olig2+ motor neurons progenitors (pMNs).

In one embodiment of the first particular aspect, the micropatterned substrate comprises a singular or plurality of polyethylene glycol (PEG) brushes or peptide-immobilizing PEG brushes arranged in a user-defined, bounded geometry.

In one embodiment of the first particular aspect, the method further comprises overlaying the neuroepithelial tissue obtained in step (c) of claim 1 with a hydrogel layer; and culturing the neuroepithelial tissue comprising the hydrogel layer for about 24 hours, whereby the tissue morphs into a bioengineered neuroepithelial tube and becomes encased in the hydrogel layer.

In a second particular aspect, the present disclosure provides an in vitro bioengineered neuroepithelial tissue obtained by the method of the first particular aspect. The tissue comprises a single rosette of polarized neuroepithelial cells and has microscale cellular organization similar to that of a transverse section of an in vivo developing human neural tube.

In a third particular aspect, the present disclosure provides a composition, comprising one or more micropatterned substrates that is capable of instructing biomimetic neural morphogenesis of cells cultured thereon. The one or more micropatterned substrates comprises at least two circular, cell-adhesive microscale regions connected by a cell-adhesive bridge.

In a fourth particular aspect, the present disclosure provides a method of producing a bioengineered neuroepithelial tube in vitro. The method includes: (a) seeding human pluripotent stem cells (hPSCs) in the presence of a Rho kinase inhibitor onto a micropatterned substrate that is capable of biomimetic neural morphogenesis of cells cultured thereon, wherein the micropatterned substrate comprises at least two circular bounded regions and connected by a cell-adhesive bridge; (b) culturing the seeded cells of step (a) on the micropatterned substrate for a first culture period of about one to two days in the presence of a pluripotency maintenance medium to obtain a first cell aggregate, wherein the pluripotency maintenance medium comprises a Rho kinase inhibitor; (c) culturing the cells of step (b) for a second culture period of about 3 to about 6 days under adherent culture conditions in a neural differentiation base medium, whereby a bioengineered neuroepithelial tissue is obtained; (d) overlaying the bioengineered neuroepithelial tissue obtained in step (c) with a hydrogel layer; (e) culturing the bioengineered neuroepithelial tissue of step (d) for about 24 hours, whereby the tissue morphs into a bioengineered neuroepithelial tube and becomes encased in the hydrogel layer; and (f) removing the hydrogel comprising the encased neuroepithelial tube from the micropatterned substrate to obtain a bioengineered neuroepithelial tube.

In a fifth particular aspect, the present disclosure provides an in vitro bioengineered neuroepithelial tube obtained by the method of the fourth particular aspect. The tube comprises a single rosette of polarized neuroepithelium and has microscale cellular organization similar to that of an in vivo developing human neural tube.

These and other features, objects, and advantages of the present invention will become better understood from the description that follows. In the description, reference is made to the accompanying drawings, which form a part hereof and in which there is shown by way of illustration, not limitation, embodiments of the invention. The description of particular embodiments is not intended to limit the invention and to cover all modifications, equivalents and alternatives. Reference should therefore be made to the claims recited herein for interpreting the scope of the invention.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

This invention can be better understood and features, aspects and advantages other than those set forth above will become apparent when consideration is given to the following detailed description thereof. Such detailed description makes reference to the following drawings, wherein:

FIG. 1 shows computer-aided designs of features presented on polydimethylsiloxane (PDMS) stamps used for microcontact printing of custom culture substrates: (top) a paired circle micropattern configuration without bridges and (bottom) a micropattern comprising pairs of circles, alternatively termed “nodes,” connected thereto by a bridge. Circle diameters vary between 150-250 μm with edge-to-edge separation of varying distances. Bridge thickness is 25 microns.

FIG. 2 (top) shows representative images of micropatterned tissue pairs across days in culture at edge-to-edge separations of 25, 50 and 75 μm without bridges. The middle graph quantifies the percentage of micropatterned tissues that fused between pairs (graph bars are D3, D4, D5, and D6 from left to right for each edge-edge distance). The bottom graph depicts the percentage of micropatterned tissue pairs that fused and formed a single rosette polarization out of all fused micropatterned tissue pairs (graph bars are D3, D4, D5, and D6 from left to right for each edge-edge distance). N=56 technical replicates per experimental group.

FIG. 3 (top) shows representative images of micropatterned tissue pairs across days in culture at edge-to-edge separations of 25, 50, 75, 100, and 125 μm with bridges. Middle graph quantifies the percentage of micropatterned tissues that fused between pairs (graph bars are D3, D4, D5, and D6 from left to right for each edge-edge distance). The bottom graph depicts the percentage of micropatterned tissue pairs that fused and formed a single rosette polarization out of all fused micropatterned tissue pairs (graph bars are D3, D4, D5, and D6 from left to right for each edge-edge distance). N=56 technical replicates per experimental group.

FIG. 4 (top) shows a single row of the micropattern design. The top middle image montage shows representative images of micropatterned neuroepithelial tissues across days in culture at edge-to-edge separations of 50 μm with bridges. The top graph quantifies the percentage of micropatterned tissues that fused across entire rows at edge-to-edge separations of 25, 50, 75, 100, and 125 μm with bridges (graph bars are D3, D4, D5, and D6 from left to right for each edge-edge distance). The bottom graph depicts the percentage of micropatterned tissue rows that fused and formed a single rosette polarization out of all fused micropatterned tissue rows (graph bars are D3, D4, D5, and D6 from left to right for each edge-edge distance). N=4 technical replicates per experimental group.

FIG. 5 shows that micropatterned neural tissue fusions span an entire row on micropatterned arrays by Day 5.

FIG. 6 illustrates that (top) circular micropatterns separated by 50 μm edge-to-edge and with bridges (middle) enable formation of a contiguous, fused tissue with a single N-cadherin⁺ rosette polarization across the entire micropatterned row (middle) that starts becoming a three dimensional tube with apical N-cadherin expression over 5 days in culture as shown with images from a confocal Z-stack (bottom).

FIG. 7 shows (top) the experimental protocol for (middle-top) deriving micropatterned forebrain neuroepithelial tubes and (middle-bottom) overlaying with Matrigel hydrogel on Day 5 for (bottom) 24 hrs.

FIG. 8 depicts representative transverse sections of forebrain neuroepithelial tubes after 24 hrs post Matrigel hydrogel overlay. Sections show (top) a central ring of polarized N-cadherin expression indicating singular neuroepithelium formation in 88% of sections, n=173 sections across 14 tissues, and (bottom) basal deposition of Laminin extracellular matrix protein.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the appended claims.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### DETAILED DESCRIPTION

In the central nervous system (CNS), the brain and spinal cord develop from a hollow tube of polarized neuroepithelial cells (NECs), also termed neural stem cells, called the neural tube. In vitro, NECs spontaneously polarize during differentiation to form similar neuroepithelial tube analogs, i.e. neural rosette structures, in both two-dimensional (2D) and three-dimensional (3D) culture. This invention provides methods for controlling neural rosette formation, thereby producing neuroepithelial tissues having an elliptical morphology with tunable aspect ratios. These elliptical neuroepithelial tissues mimic primary neurulation routinely and reproducibly form 3-D neuroepithelial tubes that exhibit structural characteristics more similar to the developing neural tube than those obtainable by previously described neural organoid derivation methods.

### I. Definitions

Unless defined otherwise, all technical and scientific terms used herein have the same meaning as commonly understood by one of ordinary skill in the art to which the invention pertains. Although any methods and materials similar to or equivalent to those described herein can be used in the practice or testing of the present invention, advantageous methods and materials are described herein.

In describing the embodiments and claiming the invention, the following terminology will be used in accordance with the definitions set out below.

As used herein, the term “human pluripotent stem cell” (hPSC) means a cell capable of continued self-renewal and of capable, under appropriate conditions, of differentiating into cells of all three embryonic germ layers. hPSCs exhibit a gene expression profile that includes SOX2⁺ and OCT4⁺. Examples of hPSCs include human embryonic stem cells (hESCs) and human induced pluripotent stem cells (hiPSCs). hESCs suitable for use in the methods disclosed herein are described in Thomson et al., 1998, Science 282:1145-1147, incorporated by reference herein in its entirety. As used herein, “iPS cells” refer to cells that are substantially genetically identical to their respective differentiated somatic cell of origin and display characteristics similar to higher potency cells, such as ES cells, as described herein. The cells can be obtained by reprogramming non-pluripotent (e.g., multipotent or somatic) cells.

As used herein, “pluripotency” means a cell's ability to differentiate into cells of all three germ layers.

As used herein, “neural stem cell” (NSC) refers to a multipotent stem cell that is PAX6⁺/Sox2⁺ and is capable of differentiating into neurons or glia of the CNS or peripheral nervous system (PNS). As used herein, neuroepithelial cells (NECs) refer to neural stem cells that are polarized epithelial cells exhibiting apico-basal polarity within neural rosette structures.

As used herein, the term “neuromesodermal progenitors” (NMPs) refers to human pluripotent stem cell-derived cells having the following gene expression profile: SOX2⁺/OCT4⁻/T⁺/PAX6⁻. NMPs are also referred to as caudal lateral epiblasts.

As used herein, the term “elliptical” means having an oval shape or shape like an ellipse, which is an elongated circle, stretched into an oval. An elliptical shape deviates from an ideal circle such that distance from the center of the shape to the edge of the shape varies (unlike in a circle where the distance from the center of the circle to any point along the edge remains constant). The terms “oval,” “elliptical” or “generally elliptical” are used interchangeably herein, and it is not considered essential that a substrate, tissue, or other material of this disclosure have a precise elliptical configuration.

As used herein, the term “neural rosette” refers to a neuroepithelial tube analog comprising neuroepithelial cells (NECs) that forms when human pluripotent stem cells are neurally differentiated in two-dimensional (2D) and three-dimensional (3D) culture. Neural rosette morphology can be elucidated by apical N-cadherin localization along constituent cell membranes with apico-basal polarity.

A “biological molecule” or “biomolecule” as used in the context of this invention refers to a molecule that is substantially of biological origin. Such molecules can include non-naturally occurring components that mimic a naturally occurring component, e.g., a non-naturally occurring amino acid.

As used herein, the terms “synthetic” and “engineered” are used interchangeably and refer to a non-naturally occurring tissue material that has been created or modified by the hand of man (e.g., formed in vitro using man-made materials) or is derived using such material (e.g., a device or composition comprising the engineered material).

As used herein, the term “organoid” refers to a tissue-like structure (i.e., exhibiting structural properties of a particular tissue type) that resembles a developing organ and is assembled in vitro by the separate addition and self-organization of cell types including, but not limited to, pluripotent stem cells or neural stem cells. See, e.g., Lancaster and Knoblich, Science 345(6194): 1247125 (2014).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “cell culture medium” as used herein (also referred to herein as a “culture medium” or “medium” or “culture media”) is a medium for culturing cells containing nutrients that maintain cell viability and support proliferation.

The term “chemically defined culture medium” or “chemically defined medium,” as used herein, means that the chemical structure and quantity of each medium ingredient is specifically known or identifiable, and can be controlled individually. As such, a culture medium is not chemically defined if (1) the chemical and structural identity of all medium ingredients is not known, (2) the medium contains unknown quantities of any ingredients, or (3) both.

As used herein, “a medium consisting essentially of” means a medium that contains the specified ingredients and those that do not materially affect its basic characteristics.

“Supplemented,” as used herein, refers to a composition, e.g., a medium comprising a supplemented component (e.g., retinoic acid or fibroblast growth factor (FGF)). For example a medium “further supplemented” with retinoic acid (RA) or a FGF, refers to the medium comprising RA or FGF, but not to the act of introducing the RA or FGF to the medium.

As used herein, “effective amount” means an amount of an agent sufficient to evoke a specified cellular effect according to this invention.

As used herein, the terms “xenogen free” and “xeno-free” are used interchangeably and refer to a material that is free of or substantially free of xenogeneic material or undefined components that are derived from a non-human source.

“Neural differentiation base medium,” as used herein, refers to a medium capable of promoting and supporting differentiation of human pluripotent stem cells towards a neural lineage, e.g., towards neuroectoderm and neuroepithelium. A neural differentiation base medium can include, but is not limited to E6 medium, as described herein and in U.S. Patent Publication No. 2014/0134732, the disclosure of which is incorporated by reference in its entirety.

The terms “purified” or “enriched” cell populations are used interchangeably herein, and refer to cell populations, ex vivo, that contain a higher proportion of a specified cell type or cells having a specified characteristic than are found in vivo (e.g., in a tissue).

As used herein, “serum-free” means that a medium does not contain serum or serum replacement, or that it contains essentially no serum or serum replacement. For example, an essentially serum-free medium can contain less than about 1%, 0.9%, 0.8%, 0.7%, 0.6%, 0.5%, 0.4%, 0.3%, 0.2% or 0.1% serum, wherein competent culturing capacity of the medium is still observed.

As used herein, “substantially free of” means that a culture medium or other composition or solution is free or nearly free of a particular component. For example, “substantially free of putrescine” means no putrescine is added to a cell culture medium above and beyond any putrescine present in the base medium, e.g., DMEM/F12. Alternatively, “substantially free of putrescine” means a final putrescine concentration less than or equal to 0.08 mg/L.

As used herein, “viability” means the state of being without substantial cell death. Pluripotent cells that are viable attach to the surface and do not stain with the dye propidium iodide absent membrane disruption. Short term viability relates to the first 24 hours after plating the cells in culture. Typically, the cells do not proliferate in that time.

As used herein, the term “cross-linking” or “cross-linked” refers to a bond that links one polymer chain to another to form, inter alia, a hydrogel. These links can be covalent bonds or ionic bonds.

The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” can mean values that are within an order of magnitude, preferably within 5-fold and more preferably within 2-fold of a particular value. Numerical quantities set forth herein are approximate unless stated otherwise, meaning that the term “about” or “approximately” can be inferred when not expressly stated.

The terms “comprising”, “comprises” and “comprised of” as used herein are synonymous with “including”, “includes” or “containing”, “contains”, and are inclusive or open-ended and do not exclude additional, non-recited members, elements, or method steps. The phraseology and terminology used herein is for the purpose of description and should not be regarded as limiting. The use of “including,” “comprising,” “having,” “containing,” “involving,” and variations thereof, is meant to encompass the items listed thereafter and additional items. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

As used in this specification and the appended claims, the singular forms “a,” “an,” and “the” include plural references unless the context clearly dictates otherwise. Any reference to “or” herein is intended to encompass “and/or” unless otherwise stated.

### II. Methods, Compositions, and Organoids

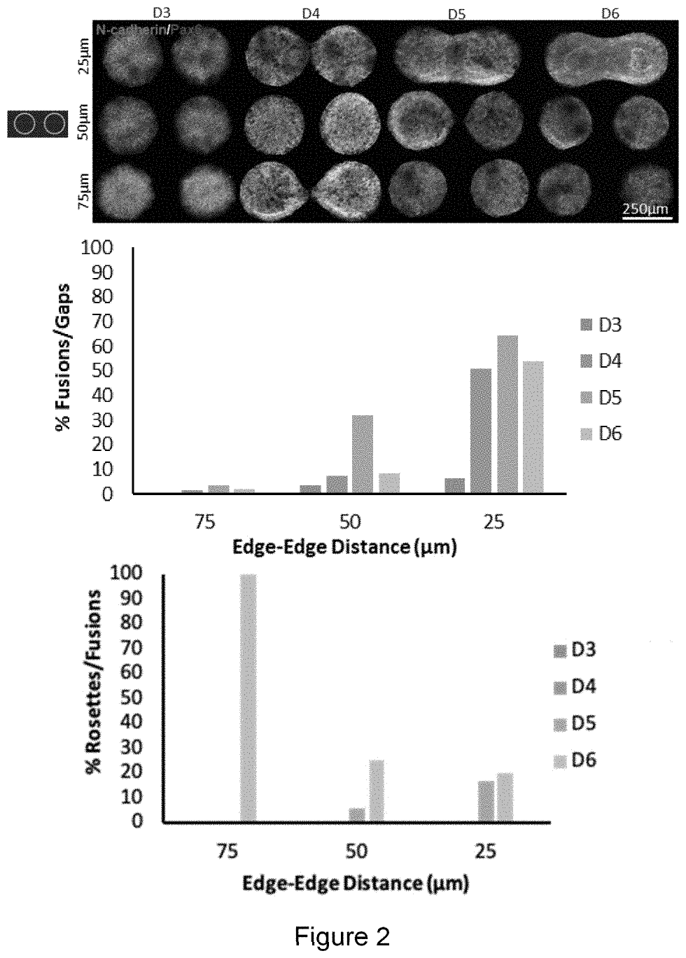

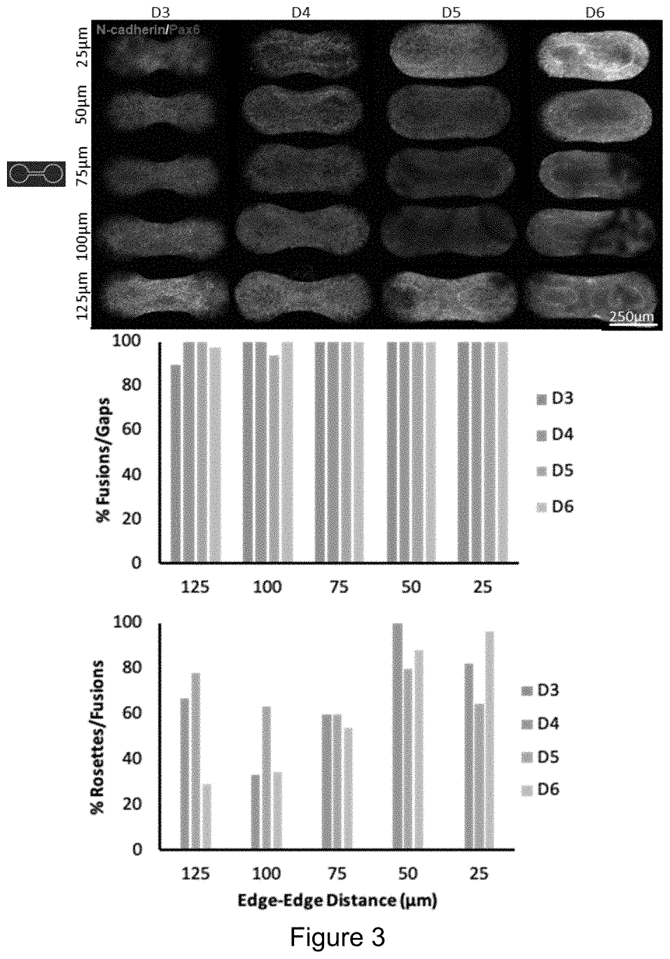

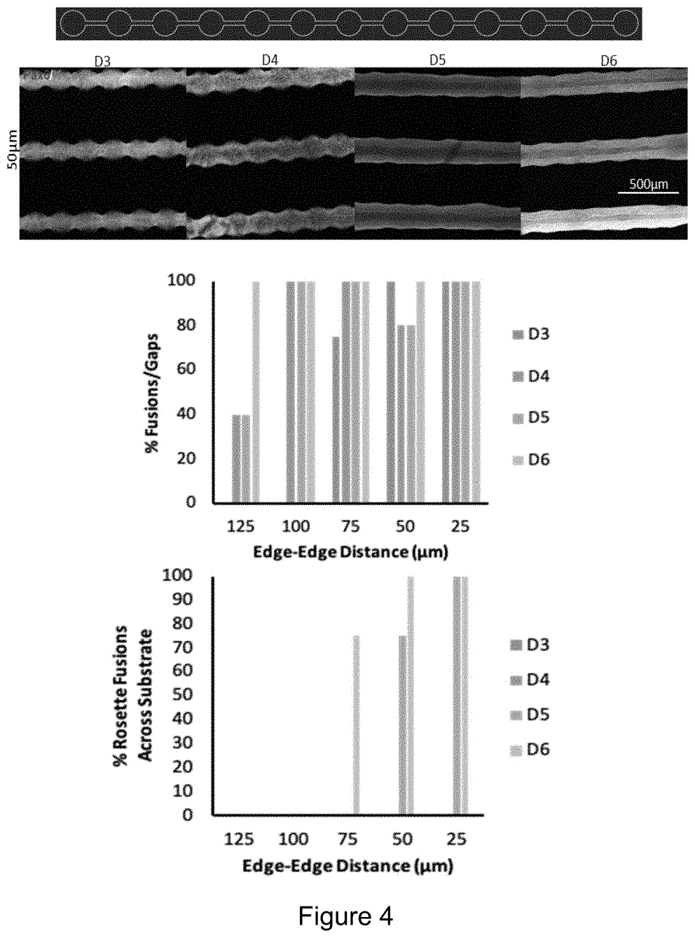

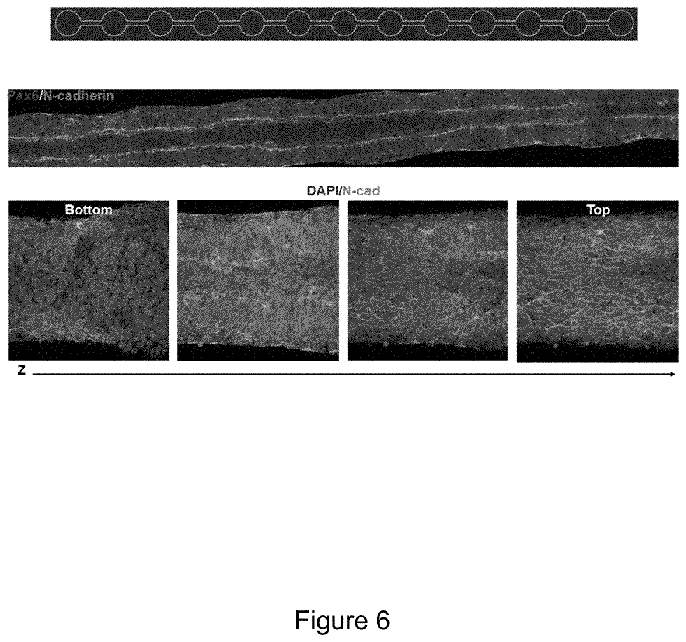

The invention set forth herein discloses a micropattern design comprising multiple circular regions connected by a cell-adhesive bridge that can induce formation of neuroepithelial tissues with a non-circular morphology and a single polarized rosette structure that spans the entire tissue. Furthermore, as shown in FIGS. 3 and 4, creating a micropattern with a connected series of multiple circles permits formation of a singular neuroepithelial rosette tissue having substantially elliptical morphology (e.g., major axis up to >1 mm and minor axis about 250-300 μm) that can be extended indefinitely. By comparison, individual circles lacking the cell-adhesive bridge (see FIG. 2) only rarely produced fused tissues or single rosettes. By directing differentiation of stem cells on micropatterned substrates of particular designs using defined conditions, in vitro organoids that are more mimetic of the in vivo neural tube structure and morphology can be obtained. Also provided by the invention set forth herein is an opportunity to model patterning of the human CNS in an in vitro 3D model, to study neural development and disease in a biomimetic in vitro human model, and to identify materials and combinatorial strategies for in vitro tissue engineering. Furthermore, extended culture of such micropatterned tissues is shown herein to recapitulate primary neurulation-like tissue folding/fusion events to form 3-D neuroepithelial tubes (FIG. 6). This capacity provides a critical step towards bioengineering neural organoids with biomimetic, reproducible anatomy. Analogous epithelial tube formation characterizes the developmental process of other human organs (e.g., the heart and digestive system), so the micropatterned culture method set forth herein could be applicable to bioengineering other tissue/organ systems.

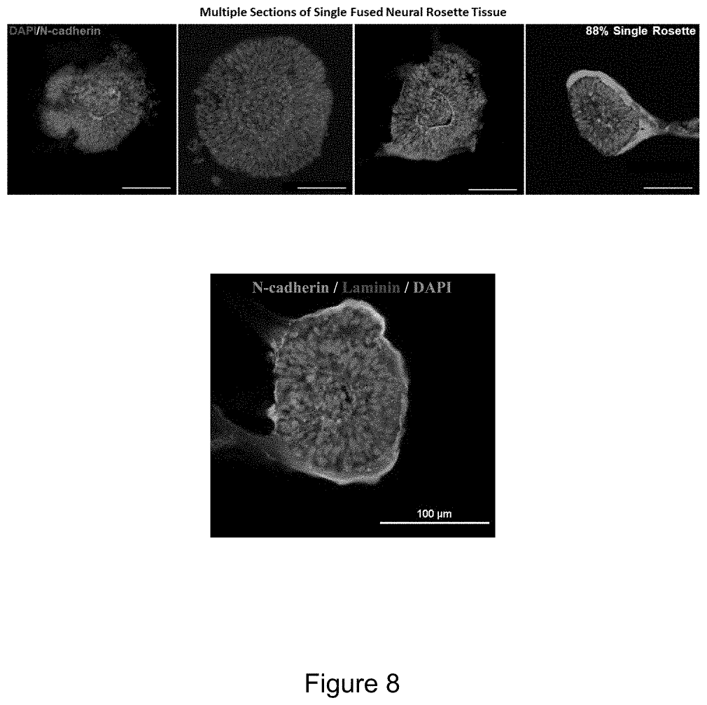

Accordingly, in a first aspect, provided herein are in vitro methods for efficiently, reproducibly, and robustly producing, from biomimetic elliptical neuroepithelial tissue, bioengineered neuroepithelial tubes having a singular neuroepithelial rosette core, where the tissue has an elliptical morphology (e.g., major axis up to >1 mm and minor axis about 250-300 μm) that can be cultured further to form an 3D tubular structure having characteristics of a nascent neural tube. The term “biomimetic elliptical neuroepithelial tissue,” as used herein refers to an elongated aggregate of cells or a tissue containing a singular neural rosette structure produced on micropatterned substrates of this disclosure, and having structural similarity to tissues found in nature, namely the anatomic structure and cytoarchitecture of the embryonic neural tube or a transverse section thereof (FIG. 3). Bioengineered neuroepithelial tubes produced therefrom are in vitro-derived (e.g., engineered) cell aggregates that, as shown in FIGS. 6 and 8, mimic polarized 3-D neural tube cytoarchitecture, with polarized NSCs displaying apical N-cadherin expression and basal extracellular matrix protein deposition along the entire axial of the 3D cylindrical tissue aggregate.

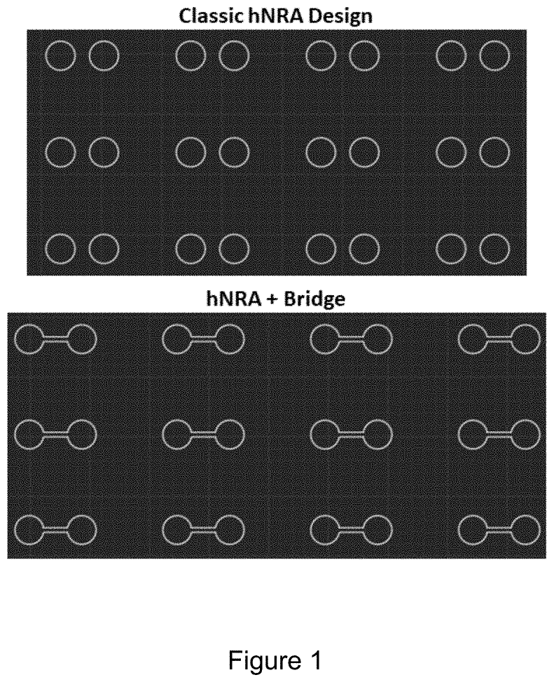

In certain embodiments, provided herein are methods for producing biomimetic elliptical neuroepithelial tissue having a singular rosette structure in vitro that comprises the following steps: seeding human pluripotent stem cells (hPSCs) in the presence of a Rho kinase inhibitor onto a micropatterned substrate that is capable of biomimetic neural morphogenesis of cells cultured thereon, wherein the micropatterned substrate comprises at least two circular bounded regions connected by a cell-adhesive bridge; culturing the seeded micropatterned substrate for a first culture period of about one to two days in the presence of a pluripotency maintenance base medium to obtain a first cell population, wherein the pluripotency maintenance base medium comprises a Rho kinase inhibitor; and culturing the cultured micropatterned substrate thereof for a second culture period of about 2 to about 6 days under adherent culture conditions in a neural differentiation base medium, whereby a biomimetic elliptical neuroepithelial tissue comprising polarized neuroepithelial cells and having microscale cellular organization similar to that of a transverse section of an in vivo developing human neural tube is obtained. Referring to FIG. 1, in certain embodiments the micropatterned substrate comprises at least two circular bounded regions connected by a cell-adhesive bridge, and in some embodiments, comprises a series of interconnected circular bounded regions. While the number of circular bounded regions should be at least two, the number of interconnected circular bounded regions in the series is limited only by the ability to prepare the predetermined arrangement on a substrate (e.g., limited by dimensions of the micropatterned substrate itself).

In some embodiments, the micropatterned substrates can be produced by simple microprinting of alkanethiols without grafted polymer brushes and can be used in the methods disclosed herein. See, Ashton et al., 2007, Stem Cells 25: 2928-2935, the disclosure of which is expressly incorporated by reference herein in its entirety. In some embodiments, the micropatterned substrate comprises an engineered, predetermined (i.e., user-defined) arrangement of one or more surface-grafted poly(ethylene glycol) (“PEG”) brushes, which robustly resist protein adsorption and thereby cell adhesion. In such embodiments, a micropatterned substrate comprises a user-defined arrangement of one or more PEG brushes grafted onto a solid support (e.g., tissue culture polystyrene, glass slide, glass, or silica substrates). Micropatterned regions resistant to cell attachment can comprise a singular or a plurality of surface-grafted PEG brushes lacking any cell attachment peptides or other moieties that promote cell attachment. Micropatterned regions that promote adherence of cells cultured thereon can comprise a singular or a plurality of PEG brushes having a peptide-immobilizing moiety. In such embodiments, the arrangement of a singular or a plurality of peptide-immobilizing PEG brushes provides a user-defined, tunable substrate that can control cell adhesion and thereby the resulting morphology of adherent cell aggregates of tissues. Furthermore, PEG brushes can be chemically modified to enable in situ conjugation of peptides, whose sequences are derived from extracellular matrix (“ECM”) proteins. For example, a micropatterned substrate can comprise an arrangement of PEG brushes comprising peptides having one or more RGD (Arg-Gly-Asp) sequence motifs, which are also known as integrin binding motifs that promote cell adhesion. A RGD sequence motif corresponds to a cell attachment site of a large number of adhesive ECM and cell surface proteins. RGD sequences are common in integrin-binding adhesion proteins such as fibronectin, collagen, and laminin. The integrin-binding activity of such adhesion proteins can be mimicked by short synthetic peptides containing the RGD sequence. Accordingly, to promote adherence of cells to particular regions of a micropatterned substrate, those regions can comprise one or more PEG brushes conjugated to RGD-containing peptides. For regions that are resistant to cell attachment, PEG brushes lacking any cell adhesion molecules (e.g., cell tethering moiety) are preferably used.

In some embodiments, a micropatterned substrate is obtained according to the synthesis protocols described by Knight et al., 2015, Chem. Commun. 51: 5238-5241, which is incorporated by reference herein as if set forth in its entirety. As described therein, a micropatterned substrate can be an azide-functionalized poly(ethylene glycol) methacrylate (PEGMA)-grafted substrate that is capable of undergoing a 1,3-dipolar cycloaddition “click” reaction with peptides conjugated to high strain molecules such as dibenzocyclooctyne (DBCO). Other peptide immobilizing PEG brushes are described by Sha et al., 2013, Biomacromolecules 14(9): 3294-3303, which is incorporated by reference herein as if set forth in its entirety. As described therein, PEG brushes present dual orthogonal chemistries (i.e., azido and acetylene groups) for ligand (e.g., peptide) immobilization via versatile copper-free click reactions, which are useful for in situ surface modifications during cell culture and thereby spatiotemporal control of adherent tissue morphologies.

In some embodiments, a micropatterned substrate comprises one or more PEG brushes or poly(ethylene glycol) methacrylate (PEGMA)-azide brushes throughout except for an array of interconnected circles having a diameter of about 100 μm, 200 μm, 250 μm, 300 μm, or more, where the circles are connected by cell-adhesive bridges. In such embodiments, when coated with extracellular matrix proteins and seeded with hPSCs, NMPs, or NSCs, the cells will adhere within the brush-free regions. When further cultured, the cells differentiate into shaped tissues confined by the surrounding inert PEG or PEGMA-azide brushes. Advantageously, the micropatterned substrates provided herein can chemically modified to display a plurality of cell adhesion peptides arranged according to user-defined, tunable spatial parameters. User-defined parameters include spacing, diameter (also sometimes referred to herein as “width”), height (also sometimes referred to herein as “length”), and number of cell adhesion peptides per unit of surface area (also referred to herein as “cell adhesion peptide surface area density”).

Accordingly, provided herein are micropatterned substrates comprising regions that promote adherence of cells cultured thereon, where these regions are engineered (i.e., configured) to have a bounded geometry to promote differentiation and morphogenesis of neuromesodermal progenitors (NMPs), neural stem cells (NSCs), or human pluripotent stem cells (hPSCs) cultured on or recruited to the matrix. As demonstrated herein, bounded geometries of a micropatterned substrate that comprises at least two circular bounded regions and connected by a cell-adhesive bridge can be used to control the macroscale structuring of neural tissue during cell differentiation and morphogenesis of hPSCs, NSCs, or NMPs on the culture substrate to form a singular neuroepithelial rosette tissue having elliptical morphology. While the bounded geometric shape can be various two-dimensional (2-D) shapes (e.g., regular or irregular) having dimensions defined by the shape (e.g., diameter, width, length and the like), it is advantageous for the micropatterned substrate to comprise a predetermined two-dimensional pattern of at least two circular regions of varying dimensions (e.g., 36 μmm, 100 μmm, 490 μmm, 4.8 mm, and 12.6 mm in diameter; typically about 150-300 μmm) that are separated from each other by a predetermined distance and are connected by a bridge of the same cell-adhesive material. In particular embodiments, the bounded geometric shape of each of the at least two circles has a diameter of about 100 μm, 150 μm, 180 μm, 200 μm, about 300 μm, or about 400 μm (e.g., about 100, 150, 200, 250, 300, 350, 400 μm, inclusive). In some embodiments, a circle or other shape having a diameter of less than 100 μm and, in some embodiments, as low as 50 μm (e.g., 50, 60, 70, 80, 90, or 100 μm, inclusive) can be used, however proliferating cells grown on smaller micropatterned surfaces (i.e., having a diameter less than 100 μm) tend to lift off the surface as an aggregate or ball of cells. Accordingly, practical constraints can set a lower limit on micropatterned surface dimensions.

With respect to the spacing between at least two circular bounded regions (spaced apart by the cell-adhesive bridge), the regions are preferably spaced apart at distance of about 25 μm to about 125 μm (e.g., about 25, 30, 35, 40, 45, 50, 55, 60, 65, 70, 75, 80, 85, 90, 95, 100, 105, 110, 115, 120, 125 μm, inclusive), and more preferably at a distance of about 50 μm to about 100 μm. Accordingly, the length of the cell-adhesive bridge that connects at least two circular bounded regions is preferably about 10 μm to about 100 μm (e.g., about 10, 15, 20, 25, 30, 35, 40, 45, 50, 55, 60, 65, 70, 75, 80, 85, 90, 95, 100 μm, inclusive), and more preferably about 20 μm to about 60 μm.

In some embodiments, the micropatterned substrate comprises, for example, a predetermined two-dimensional pattern of multiple interconnected circles in a series, where each circle has approximately the same diameter and each of any two neighboring circles shares approximately the same edge-to-edge spacing. While the diameters of each circle and spacing between circle can be varied, the inventors determined that a particular micropattern having an edge-to-edge spacing between interconnected circles of about 25 μm to about 125 μm, and more preferably about 25 μm to about 50 μm (FIGS. 3 and 4) is an advantageous configuration.

With respect to cell-adhesive bridge widths connecting at least two circular bounded regions, bridge width is advantageously about 10 μm to about 100 μm, and more specifically 25 μm.

While placement of the cell-adhesive bridge can vary, the inventors determined that it was advantageous to use a centrally located bridge. It will be understood that placement of the cell-adhesive bridge need not be at the exact center of each pair of circles to fall within the scope of this disclosure.

Any appropriate means of producing a micropatterned substrate can be used. In some embodiments, micropatterned substrates are produced by manually depositing peptide-immobilizing PEG brushes and cell adhesion peptides onto a solid support. In other embodiments, micropatterned substrates are produced using automated (e.g., robotic) techniques, microcontact printing, microfluidic etching, or deposition of various materials. In other embodiments, photolithography-based microfabrication techniques can be used. For example, photolithography-based microfabrication techniques can be used to produce templates or molds for micrometer-level patterning of a cell adhesion-resistant substance (e.g., a nonadherent agar) onto a substrate, e.g., a glass slide, a glass coverslip, or an elastomeric polymer (e.g., polydimethylsiloxane (PDMS)) coated with a cell adhesive material. In this manner, the methods disclosed herein yielded a micropatterned substrate comprising well-defined adhesive and nonadhesive domains.

As will be recognized in the art, the micropatterned substrates disclosed herein can be used in any culture system including static (e.g., tissue culture plates) and fluid flow reactor systems (e.g., microfluidic devices). Such microfluidic devices are useful for rapid screening of agents where small flow rates and small reagent amounts are required. Microfluidic devices are also useful for in vitro exposure of a micropatterned substrate to one or more gradients of transcription factors or other factors (e.g., agents to create a morphogen gradient for morphogenetic patterning of neural tissue) capable of directing or influencing neural differentiation. In another aspect, therefore, this disclosure provides is a microfluidics device comprising a micropatterned substrate as described herein for obtaining an in vitro bioengineered neuroepithelial tissue. As used herein, the term “morphogen” refers to a molecule or mixture of molecules that induces differentiation and/or proliferation of a cell such as a neural progenitor. In one embodiment, the morphogen provides spatial information via a concentration gradient that can affect patterning of a differentiating neural tissue. In some embodiments, a morphogen is a diffusible protein, cytokine, or growth factor.

The resultant bioengineered neuroepithelial tissue can have nanoscale or microscale cellular organization similar to that of the corresponding in vivo developing organ. As used herein the term “microscale cellular organization” means that structural organization of cellular components (also known as “cytoarchitecture”) of a biomimetic 3D organoid is similar to a corresponding in vivo tissue at the microscale level, meaning on the order of sizes of less than 1000 μm, and more preferably less than 100 μm.

Any suitable progenitor cells can be used to seed the micropatterned substrate. For example, pluripotent stem cells (PSCs), including embryonic stem cells (ESCs) and induced pluripotent stem cells (iPSCs) can be used. For making bioengineered neuroepithelial tubes, suitable progenitor cells are those able to form the polarized neural rosette structures (e.g., polarized staining of laminin and N-cadherin), including, but not limited to, hPSCs, neural stem cells (NSCs), or neuromesodermal progenitors (NMPs), among others.

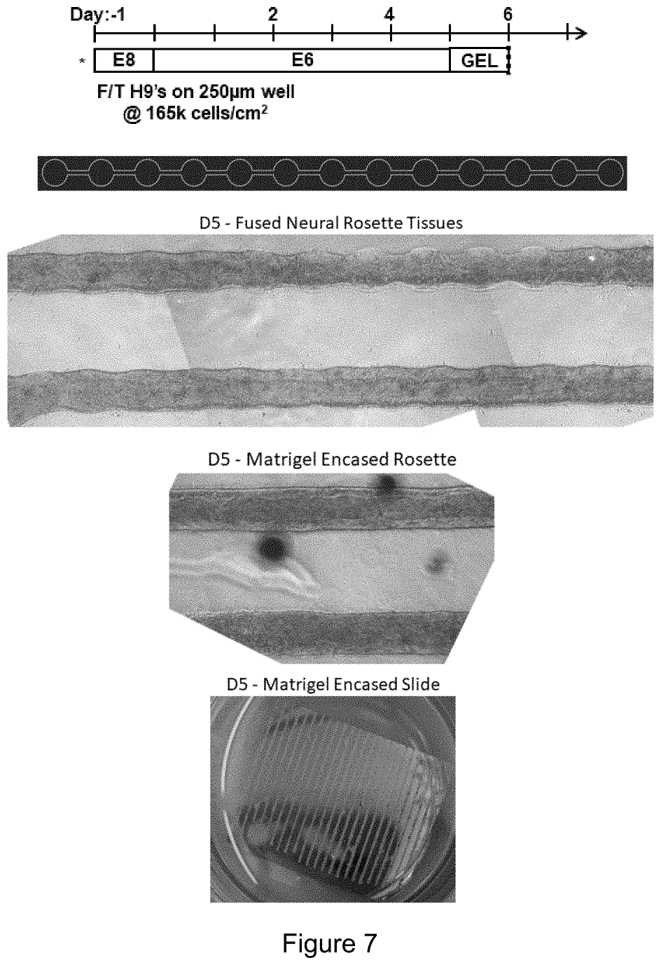

Referring to FIGS. 7 and 8, it will be advantageous in some embodiments for the methods set forth herein to further comprise overlaying a hydrogel material over the resultant biomimetic elliptical neuroepithelial tissue. In this manner, the biomimetic elliptical neuroepithelial tissue detaches from the micropatterned substrate as a three-dimensional structure encapsulated in the hydrogel. Hydrogel-encased bioengineered neuroepithelial tubes are useful for a variety of applications including, for example, bioengineering neural organoids having a 3-D structure analogous to the developing in vivo neural tube. Hydrogel-encased bioengineered neuroepithelial tubes are amenable to extended bioreactor culture or for further development and analysis including, without limitation, fixation, sectioning, immunohistochemistry (see FIG. 8), imaging, and in situ hybridization. In some embodiments, the overlaying method comprises overlaying a biomimetic elliptical neuroepithelial tissue obtained according to methods of this disclosure with a hydrogel layer; culturing the tissue comprising the overlaid hydrogel layer for about 20-52 hours (e.g., about 20, 22, 24, 26, 28, 48, 50 or 52 hours), whereby the biomimetic elliptical neuroepithelial tissue becomes encased in the hydrogel layer to yield stable bioengineered neuroepithelial tubes. In some embodiments, the hydrogel comprising the encased bioengineered neuroepithelial tube is removed from the micropatterned substrate for subsequent use or analysis. While this disclosure exemplifies using Matrigel for the hydrogel overlay, it will be understood any flexible hydrogel can be used including, without limitation, commercially available chemically defined alternatives to Matrigel substrates.

In some embodiments, it can be advantageous to fix or freeze the hydrogel containing the lifted bioengineered neuroepithelial tubes for histology or microscopy. For example, a hydrogel containing the lifted bioengineered neuroepithelial tubes can be fixed in formalin or paraformaldehyde for sectioning using routine methods. If necessary, tissues can be cleared to make transparent and more amenable to imaging using light-based microscopy. In particular, light-sheet imaging and scanning electron microscopy (SEM) is useful to detect and analyze polarization within the bioengineered neuroepithelial tubes. In exemplary embodiments, confocal or light-sheet microscopy can reveal the distribution of cell types throughout a bioengineered neuroepithelial tube produced according to the methods of the invention. In some embodiments, a three-dimensional assembly of images obtained by confocal or light-sheet microscopy is used to analyze the distribution and organization of various cells and structures therein.

In some embodiments, neural stem cells for use in the method provided herein are obtained by directed differentiation of human pluripotent stem cells (hPSCs). Suitable pluripotent cells for use herein include human embryonic stem cells (hESCs) and human iPSCs. These cells express Oct-4, SSEA-3, SSEA-4, TRA-1-60 and TRA-1-81, and appear as compact colonies having a high nucleus to cytoplasm ratio and prominent nucleolus. ESCs are commercially available from sources such as WiCell Research Institute (Madison, Wis.). As used herein, “induced pluripotent stem cells” or “iPS cells” mean a pluripotent cell or population of pluripotent cells that can vary with respect to their differentiated somatic cell of origin, that can vary with respect to a specific set of potency-determining factors and that can vary with respect to culture conditions used to isolate them, but nonetheless are substantially genetically identical to their respective differentiated somatic cell of origin and display characteristics similar to higher potency cells, such as ESCs, as described herein. See, e.g., Yu et al., 2007, Science 318:1917-1920, the disclosure of which is incorporated herein by reference in its entirety.

Induced pluripotent stem cells exhibit morphological properties (e.g., round shape, large nucleoli and scant cytoplasm) and growth properties (e.g., doubling time of about seventeen to eighteen hours) akin to ESCs. In addition, iPS cells express pluripotent cell-specific markers (e.g., Oct-4, SSEA-3, SSEA-4, Tra-1-60 or Tra-1-81, but not SSEA-1). Induced pluripotent stem cells, however, are not directly derived from embryos. As used herein, “not directly derived from embryos” means that the starting cell type for producing iPS cells is a non-pluripotent cell, such as a multipotent cell or terminally differentiated cell, such as somatic cells obtained from a post-natal individual.

Human iPS cells can be used according to a method described herein to obtain primitive macrophages and microglial cells having the genetic complement of a particular human subject. For example, it can be advantageous to obtain biomimetic elliptical neuroepithelial tissues that exhibit one or more specific phenotypes associated with or resulting from a particular disease or disorder of the particular mammalian subject. In such embodiments, iPS cells are obtained by reprogramming a somatic cell of a particular human subject according to methods known in the art. See, for example, Yu et al., 2009, Science 324:797-801; Chen et al., 2011, Nat. Methods 8(5):424-9; Ebert et al., 2009, Nature 457:277-80; Howden et al., 2011, Proc. Natl. Acad. Sci. U.S.A. 108(16):6537-42, each of which are incorporated by reference herein in its entirety.

Prior to seeding, hPSCs (e.g., hESCs or hiPSCs), can be cultured in the absence of a feeder layer (e.g., a fibroblast layer) on a substrate suitable for proliferation of hPSCs, e.g., MATRIGEL™, vitronectin, a vitronectin fragment, or a vitronectin peptide, or Synthemax®. In certain embodiments, hPSCs are passaged at least once to at least about 5 times in the absence of a feeder layer. Suitable culture media for passaging and maintenance of hPSCs include, but are not limited to, mTeSR® and E8™ media, available from Thermal Fisher/Life Technologies Inc. as Essential 8, or from Stem Cell Technologies as TeSR-E8. In some embodiments, hPSCs are maintained and passaged under xeno-free conditions, where the cell culture medium is a defined medium such as E8 or mTeSR, but the cells are maintained on a completely defined, xeno-free substrate such as vitronectin or Synthemax® (or another type-of self-coating substrate). In particular embodiments, hPSCs are maintained and passaged in E8 medium on vitronectin, a vitronectin fragment, or a vitronectin peptide or a self-coating substrate such as Synthemax®.

In certain embodiments, pluripotent stem cells that have been cultured in an adherent monolayer in a cell culture plate are subcultured onto a micropatterned surface and then cultured for a first culture period of about one (1) to two (2) days in defined pluripotency maintenance media followed by a second culture period of three (3) days to about six (6) days in a defined culture medium that supports differentiation of human pluripotent stem cells into neural stem cells, whereby PAX6-positive neural stem cells are obtained.

Once seeded, hPSCs can be cultured in differentiation media suitable to differentiate the cells into an organoid. Suitable culture medium conditions are known in the art for differentiating hPSCs or other progenitor cells into organoid tissue in vitro. In one example, the culture medium is sufficient to promote self-organization and spontaneous morphogenesis of hPSCs into engineered neuroepithelial tube organoids in vitro. Suitable media include, for example, a neural differentiation medium. Preferably, the cell-seeded hydrogels are cultured for a sufficient time to form the 3D organoid structure, for example, at least 4 days, at least 6 days, at least 8 days, or at least 16 days in culture. “Neural differentiation medium,” as used herein, refers to a medium capable of promoting and supporting differentiation of human pluripotent stem cells towards a neural lineage, e.g., towards neuroectoderm and neuroepithelium. A neural differentiation base medium can include, but is not limited to E6 medium, as described herein and in U.S. Patent Publication No. 2014/0134732, which is incorporated by reference herein in its entirety. In some embodiments, the neural differentiation medium to be used in the neural differentiation method is “E4” medium, which consists essentially of a base medium (e.g., DMEM/F12 or a similar base medium as described herein) containing water, salts, amino acids, vitamins, a carbon source, and a buffering agent, plus selenium and insulin. Optionally, the neural differentiation medium can also include ascorbate (said medium referred to herein as an “E5” medium). In some embodiments, the neural differentiation medium to be used in the neural differentiation method is “E6” medium, which consists essentially of a carbonate-buffered E5 medium plus transferrin.

As used herein, the terms “E6 culture medium” and “E6” are used interchangeably and refer to a chemically defined culture medium comprising or consisting essentially of DF3S supplemented to further comprise insulin (20 μg/mL) and/or transferrin (10.67 ng/mL). The medium can be prepared based on the formula in Chen et al., 2011, Nature Methods. 8(4), 424-429, which is incorporated by reference herein in its entirety. Similar medium is available from Thermal Fisher/Life Technologies Inc. as Essential 6, or from Stem Cell Technologies as TeSR-E6. As used herein, the terms “E8 culture medium” and “E8” are used interchangeably and refer to a chemically defined culture medium comprising or consisting essentially of DF3S supplemented by the addition of insulin (20 μg/mL), transferrin (10.67 ng/mL), human FGF2 (100 ng/mL), and human TGFβ1 (Transforming Growth Factor Beta 1) (1.75 ng/mL). The medium can be prepared based on the formula in Chen et al., 2011, Id. As an alternative, the medium is also available from Thermal Fisher/Life Technologies Inc. as Essential 8, or from Stem Cell Technologies as TeSR-E8.

In other embodiments, useful medium includes at least the same components as a neural differentiation medium mentioned above, but wherein the medium is substantially free of: a TGFβ superfamily agonist (e.g., Nodal); an albumin, and at least one of putrescine and progesterone. Optionally, a fibroblast growth factor (e.g., FGF2) can also be included in the medium. In other embodiments, the medium does not include a fibroblast growth factor. In some embodiments, a retinoic acid receptor agonist is also included to facilitate neural differentiation into certain neuronal lineages depending on the concentration of retinoid used. An exemplary class of suitable retinoic acid receptor agonists are the retinoids and retinoid analogs, which include without limitation All-Trans Retinoic Acid (ATRA), Retinol Acetate, EC23 (4-[2-(5,6,7,8-Tetrahydro-5,5,8,8-te-tramethyl-2-naphthalenyl)ethynyl)-benzoic acid; CAS No: 104561-41-3), BMS453 (4-[(1E)-2-(5,6-Dihydro-5,5-dimethyl-8-phenyl-2-naphthalenyl)ethenyl]-benzoic acid; CAS No: 166977-43-10), Fenretinide (N-(4-Hydroxyphenyl)retinamide; CAS No: 65646-68-6), AM580 (4-[(5,6,7,8-Tetrahydro-5,5,8,8-tetramethyl-2-naphthalenyl)carboxamido]benzoic acid; CAS No: 102121-60-8), Tazarotene (6-[2-(3,4-Dihydro-4,4-dimethyl-2H-1-benzothiopyran-6-yl)ethynyl]-3-pyridinecarboxylic acid ethyl ester; CAS No: 118292-40-3), and TTNPB (4-[(E)-2-(5,6,7,8-Tetrahydro-5,5,8,8-tetramethyl-2-naphthalenyl)-1-propenyl]benzoic acid; CAS No: 71441-28-6). Other exemplary retinoic receptor agonists that could be used include AC261066 (4-[4-(2-Butoxyethoxy-)-5-methyl-2-thiazolyl]-2-fluorobenzoic acid; CAS No: 870773-76-5), AC55649 (4′-Octyl-[1,1′-biphenyl]-4-carboxylic acid; CAS No: 59662-49-6), Adapalene (6-(4-Methoxy-3-tricyclo[3.3.1.13,7]dec-1-ylphenyl)-2-naphthalenecarboxylic acid; CAS No: 106685-40-9), AM80 (4-[[(5,6,7,8-Tetrahydro-5,5,8,8-tetramethyl-2-naphthalenyl)amino]carbonyl] benzoic acid; CAS No: 94497-51-5), BMS753 (4-[[(2,3-Dihydro-1,1,3,3-tetramethyl-2-oxo-1H-inden-5-yl)carbonyl]amino]benzoic acid; CAS No: 215307-86-1), BMS961 (3-Fluoro-4-[[2-hydroxy-2-(5,5,8,8-tetramethyl-5,6,7,8,-tetrahydro-2-naphthalenyl)acetyl]amino]-benzoic acid; CAS No: 185629-22-5), CD1530 (4-(6-Hydroxy-7-tricyclo[3.3.1.13,7]dec-1-yl-2-naphthalenyl) benzoic acid; CAS No: 107430-66-0), CD2314 (5-(5,6,7,8-Tetrahydro-5,5,8,8-tetramethyl-2-anthracenyl)-3-thiophenecarboxylic acid; CAS No: 170355-37-0), CD437 (6-(4-Hydroxy-3-tricyclo[3.3.1.13,7]dec-1-ylphenyl)-2-naphthalenecarboxylic acid; CAS No: 125316-60-1), and Ch55 (4-[(1E)-343,5-bis(1,1-Dimethylethyl)phenyl]-3-oxo-1-propenyl]benzoic acid; CAS No: 110368-33-7). In some embodiments, the concentration of the retinoic acid receptor agonist (e.g., all-trans retinoic acid (ATRA)) is about 0.1 μM to about 1.0 μM. A suitable concentration of retinoic acid receptor agonist ranges from about 0.1 μM to about 20 μM, e.g., about 0.2 μM, 0.3 μM, 0.5 μM, 1.0 μM, 2.5 μM, 3.0 μM, 3.5 μM, 4.0 μM, 5 μM, 7 μM, 10 μM, 12 μM, 15 μM, 17 μM or another concentration of ATRA from about 0.1 μM, to about 20 μM. In some embodiments, the concentration of ATRA is about 3.0 μM.

Guidance for directed differentiation of pluripotent stem cells to neural stem cells can be found in U.S. application Ser. No. 13/795,485 entitled “Simplified Compositions and Methods of Generating Neural Stem Cells from Human Pluripotent Stem Cells”, U.S. application Ser. No. 14/496,796 entitled “Compositions and Methods for Precise Patterning of Posterior Neuroectoderm from Human Pluripotent Stem Cells” and U.S. application Ser. No. 16/044,236 entitled “Methods And Culture Substrates For Controlled Induction Of Biomimetic Neural Tissues Comprising Singular Rosette Structures” the contents of each of which are incorporated by reference in its entirety.

Suitable progenitor cells and culture conditions are contemplated that are able to form engineered cardiac tubes or gut epithelial tubes. For example, cardiac progenitors or gut epithelial progenitor cells can be seeded onto micropatterned substrates and overlain with a supported hydrogel as described above.

The resulting bioengineered neuroepithelial tubes made by the methods of this invention have microscale cellular organization similar to that of the corresponding in vivo developing organ. For example, for bioengineered neuroepithelial tubes, the biomimetic organoids have a single contiguous, polarized neural rosette structure (e.g., as demonstrated by laminin and N-cadherin polarized staining within the neuroepithelial tube) extending, in some embodiments, along 75% of the organoid's length. In some embodiments, the contiguous, polarized neural rosette structure extends at least 75%, 80%, 90%, or 99% of the organoid's length. As used herein, the term “polarized” refers to cells having bipolar (or tripolar or greater) morphology in which certain cellular components distributed unevenly between the two (e.g., apical and basal poles) or more poles of a cell. In some embodiments, a polarized cell is a neuroepithelial cell exhibiting apico-basal polarity with respect to expression of N-cadherin. For example, the presence of apical polarity of N-cadherin foci is a surrogate marker of neural rosette formation. Polarized neuroepithelial tissue is characterized by the presence of a coherent N-cadherin ring structure (formed by apical localization of N-cadherin) toward the center of the neuroepithelial tube and laminin, a basement membrane protein, surrounding the neuroepithelial tube on the exterior surface, contacting the hydrogel. The images in FIG. 8 illustrate various immunostained transverse sections of bioengineered neuroepithelial tubes prepared according to the methods of this disclosure. Basal green fluorescence is an artifact of tissue sectioning/staining, and not equivalent to apical N-cadherin staining within the center of each section.

The methods provided herein are in vitro methods for efficiently and robustly producing engineered biomimetic elliptical neuroepithelial tissue and bioengineered neuroepithelial tubes having a singular rosette structure throughout the elongated tissue (as seen in the cross-section of the tube), where the engineered biomimetic elliptical neuroepithelial tissue exhibits microscale cellular organization (i.e., cytoarchitecture) similar to that of the developing human neuroepithelial tube. The term “biomimetic,” as used in connection with an engineered neuroepithelial tissue refers to a singular elongated tissue, preferably having an elliptical, tubular, or substantially tubular structure, having similarities to the anatomic structure and cytoarchitecture of the embryonic neuroepithelial tube in vivo (e.g., neural rosette-like cross-sectional structure) or transverse section thereof. Biomimetic elliptical neuroepithelial tissues described herein are in vitro-derived (e.g., engineered) tissues or tubes that mimic the polarized neural cytoarchitecture, with polarized NSCs displaying the apical N-cadherin expression and basal extracellular matrix protein deposition of an embryonic neural tube. As used herein, the term “substantially tubular” means that the structure has an overall tubular configuration but need not be a perfect cylinder. In some embodiments, the structure has an overall tubular configuration that has an oval or circular shape in cross-section.

In some examples, the methods disclosed herein preferably yield a bioengineered neuroepithelial tissue organoid exhibiting a polarized rosette-like cross-sectional structure in which at least 60% of cells of a cross-section of the polarized rosette structure are Pax6+/N-cadherin+ neuroepithelial cells, wherein greater than about 75% of the bioengineered 3D neuroepithelial tissue exhibits a singular, contiguous rosette structure comparable to a developing human neuroepithelial tube.

The expression (or lack thereof) of a number of cell type-associated markers can be used to characterize differentiation of hPSCs or NMPs into neural stem cells over the course of the practice of the methods described herein. For example, expression of some markers associated with pluripotency in hPSCs decline over the course of differentiation of hPSCs into neural stem cells. Such pluripotency markers include Oct4, Nanog, SSEA-3, SSEA-4, TRA-1-60, and TRA-1-81. Neuromesodermal progenitors (NMPs) have the following expression profile: SOX2⁺/OCT4⁻/T⁺/PAX6⁻. During differentiation of NMPs to neural stem cells or neuronal cell types, expression of these NMP markers and other markers associated with mesoderm or endoderm also decline over time or are absent, e.g., T (Brachyury) and SOX17. Conversely expression of markers associated with neural stem cells increases over the course of differentiation. Suitable markers (at the RNA or protein level) for neural stem cells and neural differentiation include, but are not limited to, PAX6, SOX2, Nestin, N-Cadherin, and SOX1.

In some embodiments, differentiation methods provided herein further include exposing cultured cells (e.g., cultured hPSCs) to a transient increase or “boost” of Wnt/β-catenin signaling by transiently exposing the cultured hPSCs cells to a Wnt/β-catenin signaling agonist following about 72 hours of culture, for example, as described in U.S. application Ser. No. 14/496,796, which is incorporated by reference as if set forth in its entirety. In some embodiments, cultured NMPs cells are exposed to a Wnt/β-catenin signaling “boost” following about 72 hours of culture (i.e., about 72 hours following seeding onto a micropatterned substrate). As will be appreciated by those of ordinary skill in the art, Wnt/β-catenin signaling can be activated by modulating the function of one or more proteins that participate in the Wnt/β-catenin signaling pathway to increase β-catenin expression levels or activity, TCF and LEF expression levels, or β-catenin/TCF/LEF induced transcriptional activity. In some embodiments, activation of Wnt/β-catenin signaling is achieved by inhibiting Gsk3 phosphotransferase activity or Gsk3 binding interactions. While not wishing to be bound by theory, it is believed that inhibition of Gsk3 phosphorylation of β-catenin inhibits tonic degradation of β-catenin and thereby increases β-catenin levels and activity to drive differentiation of pluripotent stem cells. Gsk3 inhibition can be achieved in a variety of ways including, but not limited to, providing small molecules that inhibit Gsk3 phosphotransferase activity, RNA interference knockdown of Gsk3, and overexpression of dominant negative form of Gsk3. Dominant negative forms of Gsk3 are known in the art as described, e.g., in Hagen et al., 2002, J. Biol. Chem., 277: 23330-23335, which describes a Gsk3 comprising a R96A mutation, which is incorporated by reference herein in its entirety.

In some embodiments, Gsk3 is inhibited by contacting a cell with a small molecule that inhibits Gsk3 phosphotransferase activity or Gsk3 binding interactions. Suitable small molecule Gsk3 inhibitors include, but are not limited to, CHIR99021, CHIR98014, BIO-acetoxime, BIO, LiCl, SB 216763, SB 415286, AR A014418, 1-Azakenpaullone, Bis-7-indolylmaleimide, and any combinations thereof. In some embodiments, any of CHIR99021, CHIR98014, and BIO-acetoxime are used to inhibit Gsk3 in pluripotent stem cells in the differentiation methods described herein. In one embodiment, the small molecule Gsk3 inhibitor is CHIR99021 at a concentration ranging from about 3 μM to about 12 μM, e.g., about 3 μM, 4 μM, 5 μM, 6 μM, 7 μM, 8 μM, 9 μM, 10 μM, 11 μM, 12 μM or another concentration of CHIR99021 from about 3 μM to about 12 μM. In another embodiment, the small molecule Gsk3 inhibitor is CHIR98014 at a concentration ranging from about 0.1 μM to about 1 μM, e.g., about 0.1 μM, 0.2 μM, 0.3 μM, 0.4 μM, 0.5 μM, 0.6 μM, 0.7 μM, 0.8 μM, 0.9 μM or another concentration of CHIR98014 from about 0.1 μM to about 1 μM. In another embodiment, the small molecule Gsk3 inhibitor is BIO-acetoxime at a concentration ranging from about 0.1 μM to about 1 μM, e.g., about 0.1 μM, 0.2 μM, 0.3 μM, 0.4 μM, 0.5 μM, 0.6 μM, 0.7 μM, 0.8 μM, 0.9 μM or another concentration of BIO-acetoxime from about 0.1 μM to about 1 μM.

In other embodiments, Gsk3 activity is inhibited by RNA interference knockdown of Gsk3. For example, Gsk3 expression levels can be knocked-down using commercially available siRNAs against Gsk3, e.g., SignalSilence® GSK-3α/βsiRNA (catalog #6301 from Cell Signaling Technology®, Danvers, Mass.), or a retroviral vector with an inducible expression cassette for Gsk3, e.g., a commercially available Tet-inducible retroviral RNA interference (RNAi) system from Clontech (Mountain View, Calif., Catalog No. 630926), or a cumate-inducible system from Systems Biosciences, Inc. (Mountain View, Calif.), e.g., the SparQ® system, catalog no. QM200PA-2.

In other embodiments, the Wnt/β-catenin signaling pathway is activated by overexpressing β-catenin itself, e.g., human β-catenin (exemplary nucleotide and amino acid sequences are found at GenBank Accession Nos: X87838 and CAA61107.1, respectively). In one embodiment, β-catenin overexpression is achieved using an inducible expression system, e.g., any of the just-mentioned inducible expression systems. Alternatively, a constitutively active, stabilized isoform of β-catenin is used, which contains point mutations S33A, S37A, T41A, and S45A as described, e.g., in Baba et al., 2005, Immunity 23(6):599-609, which is incorporated by reference herein in its entirety.

In yet other embodiments, Wnt/β-catenin signaling pathway activation in pluripotent stem cells is achieved by contacting the cells with an agent that disrupts the interaction of β-catenin with Axin, a member of the β-catenin destruction complex. Disruption of the Axin/β-catenin interaction allows β-catenin to escape degradation by the destruction complex thereby increasing the net level of β-catenin to drive β-catenin signaling. For example, the Axin/β-catenin interaction can be disrupted in pluripotent cells by contacting the cells with the compound 5-(Furan-2-yl)-N-(3-(1H-imidazol-1-yl)propyl)-1,2-oxazole-3-carboxamide (“SKL2001”), which is commercially available, e.g., as catalog no. 681667 from EMD Millipore. An effective concentration of SKL2001 to activate Wnt/β-catenin signaling ranges from about 10 μM to about 100 μM, about 20 μM, 30 μM, 40 μM, 50 μM, 60 μM, 70 μM, 80 μM, 90 μM or another concentration of SKL2001 from about 10 μM to about 100 μM.

Any appropriate method or methods can be used to confirm uniformity or the presence or absence of certain components in a biomimetic elliptical neuroepithelial tissue provided herein. Suitable methods for detecting the presence or absence of biological markers are well known in the art and include, without limitation, immunohistochemistry, qRT-PCR, RNA sequencing, and the like for evaluating gene expression at the RNA level. In some embodiments, methods such as immunohistochemistry are used to detect and identify cell types or biomolecules within a biomimetic elliptical neuroepithelial tissue. For example, whole organoids or portions thereof can be stained for specific differentiation markers by immunohistochemistry. In some embodiments, it will be advantageous to perform dual-label immunofluorescence to assess the relative expression of individual marker proteins or to detect multiple progenitor or differentiated cell types within a construct. Appropriate primary and secondary antibodies are known and available to those practicing in the art. In addition, microarray technology or nucleic acid sequencing (e.g., RNA sequencing) can be used to obtain gene expression profiles for biomimetic elliptical neuroepithelial tissues of the invention. Biological markers for neuroepithelial cells include, for example, Pax6 and N-cadherin. Quantitative methods for evaluating expression of markers at the protein level are also known in the art. For example, flow cytometry is used to determine the fraction of cells in a given cell population that express or do not express biological markers of interest.

Any appropriate method can be used to analyze structure of engineered neuroepithelial tissues and tubes obtained according to methods of this disclosure. In particular embodiments, the analytical method(s) are useful to detect the presence and identity of neurons and neuron-supporting cells (e.g., glia) in a bioengineered neuroepithelial tissue or tube. For example, confocal microscopy and other microscopy-based imaging methods can be used. In such embodiments, confocal microscopy can be used to collect multiple images of cross-sections of a biomimetic elliptical neuroepithelial tissue or bioengineered neuroepithelial tube that has been treated with detectably labeled antibodies or stains having specificity for various cell types present at particular stages of development of the human neural tube. As described in the Examples that follow, confocal images can be obtained of cross-sections of the biomimetic elliptical neuroepithelial tissue or bioengineered neuroepithelial tube fluorescently labeled to detect N-cadherin expression (FIGS. 3, 6, and 9). For example, an exemplary protocol for detecting and analyzing biomimetic elliptical neuroepithelial tissue organization includes acquiring one or more (e.g., a series of images) confocal images using a 60× objective at 1024×1024 pixels to scan through the biomimetic elliptical neuroepithelial tissue or bioengineered neuroepithelial tube in user-defined increments or steps, and analyzing a stack of the acquired confocal images to detect cell types present in or adjacent to the cellular structures using, for example, a machine learning program for image classification.

Induced pluripotent stem cell-derived engineered biomimetic elliptical neuroepithelial tissue permits modeling of drug responses in 3D structures that recapitulate neural development in an individual having, for example, a particular genetic background or detectable phenotype. Accordingly, subject-specific human iPS cell-derived biomimetic elliptical neuroepithelial tissues are useful to identify genetic factors and epigenetic influences that contribute to variable effects of a known or unknown drug on neural development/neural differentiation.

Patient-specific somatic cells for reprogramming into induced pluripotent stem cells can be obtained or isolated from a target tissue of interest by biopsy or other tissue sampling methods. In some embodiments, subject-specific cells are manipulated in vitro prior to use in a neuroepithelial tissue construct of the invention. For example, subject-specific cells can be expanded, differentiated, genetically modified, contacted to polypeptides, nucleic acids, or other factors, cryo-preserved, or otherwise modified prior to seeding on micropatterned substrates to obtain bioengineered neuroepithelial tissues described herein.

Standardizing culture conditions by using a chemically defined culture medium minimizes the potential for lot-to-lot or batch-to-batch variations in materials to which the cells are exposed during cell culture. Accordingly, the effects of various differentiation factors are more predictable when added to cells and tissues cultured under chemically defined conditions. As used herein, the term “serum-free” refers to cell culture materials that are free of or substantially free of serum obtained from animal (e.g., fetal bovine) blood. In general, culturing cells or tissues in the absence of animal-derived materials (i.e., under conditions free of xenogeneic material) reduces or eliminates the potential for cross-species viral or prion transmission.

Applications of biomimetic elliptical neuroepithelial tissues provided herein include, without limitation, in vitro screening of agents for those that modulate in vivo neural tube formation or CNS development. For example, in vitro-derived biomimetic elliptical neuroepithelial tissues can be used for high throughput screening of candidate agents. Standardized, reproducible production of biomimetic elliptical neuroepithelial tissues can provide a revolutionary experimental paradigm for conducting personalized neuroscience studies. For example, neuroepithelial organoid tissues are useful to study the effects of genetic mutations on development and function across the human CNS, to conduct personalized neuroscience studies using neural stem cells derived from induced pluripotent stem cells (iPS cells) of a particular human subject, and to assess neurotoxicity of various agents or other effects on neural development. In some embodiments, engineered neuroepithelial tissues of the invention are useful in drug discovery and development including screening for metabolic stability, drug-drug interactions, toxicity and infectious disease. Exemplary test agents include, without limitation, infectious agents, proteins, peptides, antibodies, small molecules, oligonucleotides, polynucleotides, peptidomimetics, cytotoxic agents, pharmaceutical agents, and xenobiotics (e.g., environmental toxin, chemical/biological warfare agent, a natural compound, and a nutraceutical).

In some embodiments, biomimetic elliptical neuroepithelial tissues as described herein can be screened to identify agents that modulate neural tube development and development of the human CNS. Screening methods can comprise or consist essentially of contacting a test agent to in vitro derived bioengineered neuroepithelial tissues; and detecting an effect of the agent on bioengineered neuroepithelial tissues (e.g., disrupt or otherwise alter development of the biomimetic elliptical neuroepithelial tissues or differentiation of neural cell types within a biomimetic elliptical neuroepithelial tissue). In some embodiments, screening methods include screening candidate compounds to identify test agents that promote the development of the human CNS. In other embodiments, candidate compounds can be screened for toxicity to human neural cell types or tissues. In some embodiments, detecting comprises detecting at least one positive or negative effect of the agent on morphology or life span of such cells and tissues, whereby an agent that increases or reduces the life span of human neural cell types or tissues, or has a positive or negative impact on the morphology of human neural cell types or tissues, is identified as having an effect on development of the human neuroepithelial tube or neural tissues. In some embodiments, detecting comprises performing a method including but not limited to RNA sequencing, gene expression profiling, transcriptome analysis, cell proliferation assays, metabolome analysis, detecting reporter or sensor, protein expression profiling, Förster resonance energy transfer (FRET), metabolic profiling, and microdialysis. In some embodiments, the agent can be screened for an effect on gene expression, and detecting can comprise assaying for differential gene expression relative to an uncontacted biomimetic elliptical neuroepithelial tissue. In addition, in some embodiments, biomimetic elliptical neuroepithelial tissues of this disclosure are suitable as direct transplants for tissue regeneration and repair.

In exemplary embodiments, detecting and/or measuring a positive or negative change in a level of expression of at least one gene following exposure (e.g., contacting) of a test compound to one or more biomimetic elliptical neuroepithelial tissues comprises whole transcriptome analysis using, for example, RNA sequencing. In such embodiments, gene expression is calculated using, for example, data processing software programs such as Light Cycle, RSEM (RNA-Seq by Expectation-Maximization), Excel, and Prism. See Stewart et al., PLoS Comput. Biol. 9:e1002936 (2013), which is incorporated by reference herein in its entirety. Where appropriate, statistical comparisons can be made using ANOVA analyses, analysis of variance with Bonferroni correction, or two-tailed Student's t-test, where values are determined to be significant at P<0.05. Any appropriate method can be used to isolate RNA or protein from neural constructs. For example, total RNA can be isolated and reverse transcribed to obtain cDNA for sequencing.

Test compounds can be dissolved in a solvent such as, for example, dimethyl sulfoxide (DMSO) prior to contacting to one or more bioengineered neuroepithelial tissues provided herein. In some embodiments, identifying agents comprises analyzing the contacted bioengineered neuroepithelial tissues for positive or negative changes in biological activities including, without limitation, gene expression, protein expression, cell viability, and cell proliferation. For example, microarray methods can be used to analyze gene expression profiles prior to, during, or following contacting the plurality of test compounds to the bioengineered neuroepithelial tissues. In some embodiments, a method of the present invention further comprises additional analyses such as metabolic assays and protein expression profiling.

In another aspect, provided herein is a cell culture substrate comprising one or more components useful for obtaining an in vitro biomimetic elliptical neuroepithelial tissue or a bioengineered neuroepithelial tube. Components of the cell culture substrate can include one or more micropatterned substrates as described herein. The cell culture substrate can be a tissue culture dish, well-plate (e.g., multi-well plate), microfluidic device, or any other substrate suitable for culturing cells on the one or more micropatterned substrates to obtain an in vitro biomimetic neuroepithelial tissue.

In another aspect, provided herein is a kit comprising one or more components useful for obtaining an in vitro biomimetic neuroepithelial tissue or a bioengineered neuroepithelial tube. Components of the kit can include one or more micropatterned substrates as described herein. The kit can also contain a chemically defined culture medium and one or more medium additives (e.g., growth factors, small molecule compounds, culture medium supplements). The kit can further contain progenitor cells useful for the seeding of the micropatterned substrate and instructions for cell seeding and culture. In some embodiments, it will be advantageous for a kit to comprise one or more micropatterned substrates as described herein and one or more agents (e.g., growth factors, small molecule agents, agents to create a morphogen gradient) useful to further differentiate the neuroepithelial tissue to contain various types of neurons. In such embodiments, the kit can also comprise a microfluidics device or components thereof, for example, to deliver patterning agents to the bioengineered neuroepithelial tissue in a pre-determined manner. In some embodiments, the kit further comprises a hydrogel suitable for use as an overlay on the biomimetic elliptical neuroepithelial tissue to obtain an encased bioengineered neuroepithelial tube according to methods of this disclosure.

The invention will be more fully understood upon consideration of the following non-limiting Examples.

### EXAMPLE

This Example demonstrates in vitro production of a biomimetic elliptical neuroepithelial tissue that contains a single polarized rosette structure that spans the length of the tissue (see FIGS. 3, 4, 5, and 6). As demonstrated in FIG. 8, cross-sections of bioengineered neuroepithelial tubes produced therefrom revealed a well-organized single neural rosette of polarized N-cadherin-expressing cells spanning the length of the tissue.

### Materials and Methods

Micropatterned array substrate fabrication: Micropatterned array cell culture substrates were fabricated using a combination of methods known in the art (e.g., Knight et al., 2015, Chemical Communications 51:5238-241; Sha et al., 2013, Biomacromolecules 14: 3294-3303, which are incorporated by reference herein in their entirety). Polydimethylsiloxane (PDMS) stamps with arrays of post and micro-well features were generated as relief molds of silicon wafers. (FIG. 1). The wafers were designed in AutoCAD and purchased from FlowJEM (available at flowjem.com). PDMS stamps were coated with w-mercaptoundecyl bromoisobutyrate (2 mM in 100% ethanol), dried under inert gas, and then brought in conformal contact with glass coverslips coated with 180 nm gold (Au) atop 30 nm titanium (Ti). Micropatterned slides were then incubated in 100% ethanol for 10 min, prior to being dried under nitrogen and transferred to a Schlenk flask under vacuum. A solution of poly(ethylene glycol) methyl ether methacrylate (PEG-MEMA) macromonomer (Sigma Aldrich) with water, methanol (obtained from Thermo Fisher), copper(II) bromide (Sigma Aldrich), and 2′,2-bipyridine (Sigma Aldrich) was degassed and transferred to the reaction flask. Surface-initiated atom-transfer radical-polymerization (SI-ATRP) of PEG polymers was initiated by injection of L-ascorbic acid (Sigma Aldrich) in deionized water into the reaction flask. ATRP was allowed to continue for 16 hours at room temperature to generate micropatterned PEG brushes. Polymerization was terminated via addition of air and followed by rinsing with ethanol and water before drying under inert gas. In a sterile hood, the substrates were rinsed five times with sterile PBS (Thermo Fisher) and transferred to individual wells of a 12-well tissue-culture polystyrene (TCPS) plate where they were rendered cell-adhesive through adsorption of 0.083 mg/mL Matrigel (WiCell) in DMEM/F-12 (Thermo Fisher) via overnight incubation at 37° C. (FIG. 2).

Results: showing generation of single fused neural rosette tissue and neuroepithelial tissue, including neuroepithelial tubes are described in detail below and in corresponding FIGS. 2-8, as provided below:

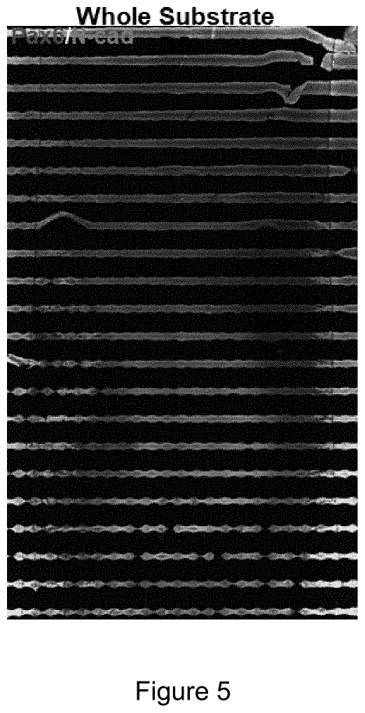

Generation of micropatterned forebrain neuroepithelial tissues: WA09 (H9) hESCs were obtained from, authenticated as karotypically normal, and tested negative for mycoplasma by WiCell. All pluripotent lines were maintained in Essential eight medium (E8) on Matrigel-coated TCPS plates and routinely passaged with Versine (Thermo Fisher) for no more than 10 passages. NEC derivation from hPSCs was performed in accordance with the E6 protocol (Lippmann et al., 2014, Stem Cells 32:1032-1042, which is incorporated by reference herein in its entirety). To generate hPSC-derived micropatterned tissues, hPSC cultures at ˜85% confluency were rinsed with PBS, dissociated with Accutase for 5 min at 37° C. and collected via centrifugation at 1000 rpm for 5 min. (FIGS. 2-3). Singularized hPSCs were then suspended in E8 media with 10 μM ROCK inhibitor and seeded onto micropatterned substrates at 165,000 cells/cm² in 2 mL of media per well in a 12-well plate. The following day the media was replaced with 2 mL of E6 media and 50% media changes were performed daily thereafter. (FIG. 4). By Day 5, micropatterned neural tissue fusions spanned an entire row on the micropatterned arrays. (FIG. 5). Circular micropatterns separated by 50 μm from edge-to-edge and with bridges formed a contiguous, fused tissue with a single N-cadherin⁺ rosette polarization across the entire row of micropatterned array, starting to form a three dimensional tub with apical N-cadherin expression over 5 days in culture. (FIG. 6).

Matrigel encapsulation of micropatterned forebrain neuroepithelial tissues: Upon tissue fusion and rosette alignment (Day 5 or 6), 750 μL neuroepithelial tissue media was removed using a P1000 micropipette and replaced with 750 μL of Matrigel at 4° C. (FIG. 7). Matrigel gelation was then conducted via incubation at 37° C. for 20 min. (FIG. 7). Once gelled, 1 mL of E6 media is pipetted into each well and placed back in the incubator. The following day, sterile forceps were used to detach the layer of Matrigel from the walls of the tissue culture plate. (FIG. 8). Fresh E6 media, pipetted underneath the gel separated the Matrigel layer from micropatterned substrates. (FIG. 8). The substrates were then removed delicately with tweezers and discarded. 50% E6 media changes were performed every day thereafter until the desired experimental timepoints. (FIG. 8).

The invention has been described in connection with what are presently considered to be the most practical and particular embodiments. However, the present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

We claim:

1. A method of producing a biomimetic elliptical neuroepithelial tissue having a singular rosette structure in vitro, the method comprising:
(a) seeding human pluripotent stem cells (hPSCs) in the presence of a Rho kinase inhibitor onto a micropatterned substrate that is capable of biomimetic neural morphogenesis of cells cultured thereon, wherein the micropatterned substrate comprises at least two circular bounded regions connected by a cell-adhesive bridge;
(b) culturing the seeded cells of step (a) on the micropatterned substrate for a first culture period of about one to two days in the presence of a pluripotency maintenance medium to obtain a first cell aggregate, wherein the pluripotency maintenance medium comprises a Rho kinase inhibitor; and
(c) culturing the cells obtained in step (b) for a second culture period of about 3 to about 6 days under adherent culture conditions in a neural differentiation base medium,
whereby a biomimetic elliptical neuroepithelial tissue having a singular rosette structure is obtained, wherein the tissue comprises polarized neuroepithelial cells and has a microscale cellular organization similar to that of a transverse section of an in vivo developing human neural tube.

2. The method of claim 1, wherein each of the at least two circular bounded regions has a diameter of about 100 μm to about 300 μm.

3. The method of claim 1, wherein the cell-adhesive bridge has a length of about 25 μm to about 125 μm, and has a width of about 10 μm to about 50 μm.

4. The method of claim 1, wherein the hPSCs are seeded onto the micropatterned substrate at a density of between about 75×10³ cells/cm² and about 2.5×10⁵ cells/cm².

5. The method of claim 1, wherein the pluripotency maintenance medium is a chemically defined medium comprising DMEM/F-12, ascorbic acid, sodium bicarbonate, selenium, insulin, transferrin, FGF2, and TGFβ1.

6. The method of claim 1, wherein the pluripotency maintenance medium is E8 medium.

7. The method of claim 1, wherein the neural differentiation base medium is a chemically defined medium comprising DMEM/F-12, ascorbic acid, sodium bicarbonate, selenium, insulin, and transferrin.

8. The method of claim 1, wherein the neural differentiation base medium is E6 medium.

9. The method of claim 8, wherein the neural differentiation base medium further comprises one or more of an FGF with or without an activator of β-catenin pathway signaling.

10. The method of claim 9, wherein the FGF is FGF2, FGF8a, FGF8b, FGF8f, FGF17, or FGF18.

11. The method of claim 9, wherein the activator of β-catenin pathway signaling is a GSK3 kinase inhibitor.

12. The method of claim 11, wherein the GSK3 kinase inhibitor is CHIR99021.

13. The method of claim 1 further comprising transiently exposing cells on the micropatterned substrate to an activator of Wnt/β-catenin signaling about 24-72 hours after plating onto the micropatterned substrate.

14. The method of claim 1 further comprising about 24-72 hours after seeding cells onto the micropatterned substrate, exposing the seeded cells to RA and Sonic Hedgehog (SHH) or a SHH signaling agonist for about 1 to about 5 days, whereby the rosette structure comprises Olig2+ motor neurons progenitors (pMNs).

15. The method of claim 1, wherein the micropatterned substrate comprises a singular or plurality of polyethylene glycol (PEG) brushes or peptide-immobilizing PEG brushes arranged in a user-defined, bounded geometry.

16. The method of claim 1 further comprising:
overlaying the neuroepithelial tissue obtained in step (c) of claim 1 with a hydrogel layer; and
culturing the neuroepithelial tissue comprising the hydrogel layer for about 24 hours, whereby the tissue morphs into a bioengineered neuroepithelial tube and becomes encased in the hydrogel layer.

17. An in vitro bioengineered neuroepithelial tissue obtained by the method of claim 1, the tissue comprising a single rosette of polarized neuroepithelial cells and having microscale cellular organization similar to that of a transverse section of an in vivo developing human neural tube.

18. A composition, comprising:
one or more micropatterned substrates that is capable of instructing biomimetic neural morphogenesis of cells cultured thereon,
wherein the one or more micropatterned substrates comprises at least two circular, cell-adhesive microscale regions connected by a cell-adhesive bridge.

19. A method of producing a bioengineered neuroepithelial tube in vitro, the method comprising:
(a) seeding human pluripotent stem cells (hPSCs) in the presence of a Rho kinase inhibitor onto a micropatterned substrate that is capable of biomimetic neural morphogenesis of cells cultured thereon, wherein the micropatterned substrate comprises at least two circular bounded regions and connected by a cell-adhesive bridge;
(b) culturing the seeded cells of step (a) on the micropatterned substrate for a first culture period of about one to two days in the presence of a pluripotency maintenance medium to obtain a first cell aggregate, wherein the pluripotency maintenance medium comprises a Rho kinase inhibitor;
(c) culturing the cells of step (b) for a second culture period of about 3 to about 6 days under adherent culture conditions in a neural differentiation base medium, whereby a bioengineered neuroepithelial tissue is obtained;
(d) overlaying the bioengineered neuroepithelial tissue obtained in step (c) with a hydrogel layer;
(e) culturing the bioengineered neuroepithelial tissue of step (d) for about 24 hours, whereby the tissue morphs into a bioengineered neuroepithelial tube and becomes encased in the hydrogel layer; and
(f) removing the hydrogel comprising the encased neuroepithelial tube from the micropatterned substrate to obtain a bioengineered neuroepithelial tube.

20. An in vitro bioengineered neuroepithelial tube obtained by the method of claim 19, the tube comprising a single rosette of polarized neuroepithelium and having microscale cellular organization similar to that of an in vivo developing human neural tube.
